(19) United States
(12) Patent Application Publication
(10) Pub. No.: US 2014/0364489 A1
(43) Pub. Date: Dec. 11, 2014

(54) Pharmaceutical Composition Producing antioxidant, antimicrobal, antitoxic protein - human lactoferrin, production process and therapeutic method.

(71) Applicant: Obschestvo s ogranichennoi otvetstvennostyu "NTpharma", Moscow (RU)

(72) Inventors: Maksim M. SHMAROV, Moscow (RU); Raisa I. YAKUBOVSKAYA, Moscow (RU); Rustam R. ATAULLAKHANOV, Moscow (RU)

(21) Appl. No.: 14/469,749

(22) Filed: Aug. 27, 2014

Related U.S. Application Data
(63) Continuation of application No. PCT/RU2012/000365, filed on May 11, 2012.

(30) Foreign Application Priority Data
Feb. 28, 2012 (RU) 2012107032

(51) Int. Cl. A61K 38/48; A61K 48/00

(52) U.S. Cl. CPC A61K 38/482; A61K 48/0075
USPC 514/44 R; 435/91.1

(57) ABSTRACT

The pharmaceutical composition producing the antioxidant, antimicrobal, antitoxic protein—human lactoferrin in which the therapeutic effect is achieved as a result of the antioxidant, antimicrobal, antitoxic protein—human lactoferrin on the human body different in what it contains human adenovirus 5 genome based non-replicating nanoparticles with the human lactoferrin gene insert expressing human lactoferrin in a therapeutically effective amount in the body and containing the formulating buffer. The non-replicating nanoparticles content makes no less than 2.33×10¹¹ virus particle (v.p.) per ml of the formulating buffer.

### RELATED APPLICATIONS

This Application is a Continuation application of International Application PCT/RU2012/000365, filed on May 11, 2012, which in turn claims priority to Russian Patent Applications No. RU 2012107032, filed Feb. 28, 2012, both of which are incorporated herein by reference in their entirety.

### FIELD OF THE INVENTION

The invention belongs to the sphere of medicine, in particular, to toxicology and radiology, to antioxidant protein-based medications and their application methods.

### BACKGROUND OF THE INVENTION

There is a known pharmaceutical composition containing human lactoferrin inducing various physiological changes, immunomodulating effects, reduction of inflammatory reactions and solid tumour inhibition in the human body. (U.S. Pat. No. 6,333,311).

There is a known pharmaceutical composition containing recombinant human lactoferrin (U.S. Pat. No. 5,955,316). There is a composition intended for therapy of diabetic ulcers for application, peroral or parenteral use (U.S. Pat. No. 7,524,814).

A method to prevent and to treat grave post-operative complications (pyoinflammatory and post-hemorrhagic) with polyorgan insufficiency and general intoxication manifestations by means of daily intravenous, intracavitary, intratracheal administration of human lactoferrin and human ceruloplasmin medications (Russian Patent No. 2199337) is known.

Reasons complicating achievement of stable therapeutic effect after use of the above-stated compositions are isolated and purified proteins acting as active components that in any administration methods are quickly removed from the body of the patient, which preconditions the necessity of repeated introduction of pharmaceutical compositions containing the antioxidant protein lactoferrin in order to maintain the therapeutically effective concentration in the body. In addition to difficulty from the medical point of view, it isn't economically sound.

Thus, the urgent necessity in development of pharmaceutical compositions and methods of prevention and treatment on their basis that would be therapeutically highly efficient, economically justified and required less time and labour costs of medical staff when applying them arose.

The actively developing trend of gene-therapeutic recombinant adenovirus-based medications is known. Among human adenoviruses the human adenovirus serotype 5 (Ad5) is characterized to the fullest extent (Zaia J. A. The status of gene vectors for the treatment of diabetes, Cell Biochem, Biophys, 2007, v. 48(2-3), p. 183-90).

One of the recombinant adenovirus advantages is a long-term high target gene expression, up to 28 days, and in some laboratory mice this duration is longer (up to 60-90 days) (S. L. Brody, R. G. Crystal, Adenovirus-Mediated in Vivo Gene Transfer, Annals new york academy of sciences, 1994, 716, p. 90-103).

Successful application of the recombinant adenovirus-based substance expressing human lactoferrin in experiments on mice bearing breast cancer is known. The substance injection in the tumour tissue resulted in its growth inhibition induced by cell apoptosis (Inhibition of tumor growth by recombinant adenovirus containing human lactoferrin through inducing tumor cell apoptosis in mice bearing EMT6 breast cancer, Wang J., Li Q., Ou Y., Han Z., Li K., Wang P., Zhou S., Arch Pharm Res, 2011, Vol 34, No 6, p. 987-995).

But this medication doesn't provide antioxidant and antimicrobial lactoferrin effect as the described therapeutic method ensures only its antineoplastic action.

The adenovirus is known for efficient expression of human lactoferrin around neoplastic cells demonstrating stable uterine cervix cancerous growth inhibition, immune system reinforcement and has no toxic side effects (Patent Application No. 200910021705, 19 Aug. 2009, Li Q., Li J., Han Z., Wu G., Hu J., Recombinant adenovirus carrying human lactoferrin, preparation and uses thereof, China).

This medication has a narrow antineoplastic action as the described method of its application around neoplastic cells doesn't release antioxidant, antimicrobial and antitoxic lactoferrin action.

The antibacterial, antioxidant, detoxyfying, immunomodulating and anticarcinogenic medication carrying human lactoferrin isolated from the human milk as the active substance (Russian Patent No. 2165769) is the closest pharmaceutical composition of the same intended use to the claimed invention by a combination of characteristics and is taken as a prototype. The dosage form of the known medication is a lyophilisate containing from 10 to 90% of human lactoferrin. The medication is intended for prevention and/or treatment of infectious, inflammatory diseases and toxic states of various etiology.

Reasons complicating achievement of the stable therapeutic effect for the above-stated medication is the necessity of numerous medication infusions due to quick excretion of lactoferrin from the body and consequently high costs of medical equipment and labour costs of medical staff for achievement of the required therapeutic effect.

The unique nature and deficit of raw material—human milk—seriously restricts scaling of the medication production and consequently a possibility of its use in required amount for medical indications.

### SUMMARY OF THE INVENTION

The task of this invention is to develop a pharmaceutical composition producing the antioxidant, antimicrobal, antitoxic protein—human lactoferrin—providing stable therapeutic effect after single injections of this composition, reduction of the medication consumption, use of medical tools and the labour costs of medical personnel in order to achieve the required therapeutic result, methods to receive it and a therapeutic method by means of exposure of the human body to this pharmaceutical composition.

The formulated challenge is solved as the pharmaceutical composition, therapeutic effect of which is achieved through antioxidant, antimicrobial and antitoxic action of the human lactoferrin, contains human adenovirus serotype 5 based non-replicating nanoparticles with a human lactoferrin insert expressing human lactoferrin in a therapeutically effective amount in the body, containing a formulating buffer at the same time. The content of non-replicating nanoparticles is no less than 2.33×1011 virus particle (v.p.) per ml of the formulating buffer. A production process of the pharmaceutical composition containing human adenovirus serotype 5 based non-replicating nanoparticles with an insert of the expressing cassette including a promoter, a human lactoferrin gene and a polyadenylation signal, involves achieving the target activity by means of the permissive cell culture seeding with 293 non-replicating nanoparticles carrying the human lactoferrin gene and growth of non-replicating nanoparticles in the cell until they achieve the required content, multi-stage purification by centrifuging, four-fold freezing-thawing in the buffer solution of the solid part obtained at the previous stage, additional treatment by nuclease with subsequent isolation of non-replicating nanoparticles carrying human lactoferrin gene from damaged cells by centrifuging and subsequent extraction of the obtained supernatant. The subsequent purification is done by ultrafiltration requiring dissolution of the obtained supernatant by the buffer and mixing with filtering under pressure and purification by means of anion-exchange chromatography and then—by exclusive chromatography. Add ethanol and ethylene-tetraacetic acid to the obtained eluate, after which perform regular filtration and then obtain the finished product by dissolution of the product obtained at the previous stage by a formulating buffer until the required amount of non-replicating nanoparticles is achieved. Perform the construction of non-replicating nanoparticles with the human lactoferrin gene insert by a homologous recombination method in the cell culture. Take the therapeutically effective dosage of non-replicating nanoparticles per 3 ml of the final composition volume. The dosage form is 1 ml, 2 ml and 3 ml. A therapeutic method involves introduction of the pharmaceutical composition producing the antioxidant, antimicrobal, antitoxic protein—human lactoferrin—specified in the application. The pharmaceutical composition is injected to the patient intravenously. The pharmaceutical composition might be administered by intravenous infusion. Administer the pharmaceutical composition for therapy of toxic states induced by pyoinflammatory diseases caused by various microorganisms, physical effects and chemical agents, including means of drug therapy and X-ray therapy. A therapeutically effective dosage of non-replicating nanoparticles with the human lactoferrin gene insert constitutes from 7×1011 virus particle (v.p.) to 7×1013 virus particle (v.p.) per person. Perform administration of the pharmaceutical composition sequentially in two stages. Administer the pharmaceutical composition at the second stage in a day after the first administration. Administer the pharmaceutical composition at the first stage in the volume containing ⅓ of the full therapeutic al dosage of the pharmaceutical composition, and at the second stage introduce the remaining ⅔ of the full therapeutic al dosage of the pharmaceutical composition. Dissolve ⅓ of the full therapeutic al dosage of the pharmaceutical composition in 66 ml of the physiologically acceptable diluent and ⅔ of the complete dosage dissolve in 134 ml of the physiologically acceptable diluent. Glucose solution is a physiologically acceptable diluent. Glucose solution concentration might be either 5% or 10%.

The above-stated single technical, therapeutic and economic results on application of the invention under the claimed invention are achieved due to the fact that the claimed method, as well as the known method to prevent and/or to treat inflammatory and/or infectious diseases and/or toxic states is implemented with the help of the antioxidant protein lactoferrin. The peculiarity of the claimed method involved production of the antioxidant protein occurs directly in the human body following administration of the non-replicating nanoparticles with insert of the antioxidant protein gene and not through multiple administrations in the form of the proteins isolated from the natural source. The expressed protein renders therapeutic effect as the antimicrobial and/or antiinflammatory and/or immunomodulating and/or antioxidant and/or detoxifying and/or anticancerous agent.

The non-replicating nanoparticles based pharmaceutical composition producing the antioxidant human protein lactoferrin is administered at following dosages in order to achieve the therapeutic effect:

for therapy of toxic states caused by pyoinflammatory diseases induced by various microorganisms, physical effects and chemical agents, including drug and X-ray therapy, from 7×1011 to 7×1013 virus particle (v.p.) per person;

for prevention of post-injection complications in the beginning of the therapy at the first stage of—administer ⅓ of the full therapeutic dosage of non-replicating nanoparticles carrying the human lactoferrin gene;

for prevention of post-injection complications in the beginning of the second stage—administer ⅔ of the full therapeutic dosage of non-replicating nanoparticles carrying the human lactoferrin gene;

The essence of the invention involves the following. The claimed pharmaceutical composition contains non-replicating nanoparticles with insert of the gene expressing the antioxidant human protein—lactoferrin as the main active component. This protein exerts comprehensive biological properties predetermining its antioxidant, detoxifying, immunomodulating, antimicrobial and anticancerous action in the human body. The major significant difference of the claimed pharmaceutical composition from the previously known pharmaceutical composition is the fact that the antioxidant protein is produced on a long-term basis following a single administration of the nano-structure directly in the body but is not introduced many times in the body in the form of the protein isolated from any source. The pharmaceutical composition is compatible with any pharmaceutically suitable solvents. The claimed range of the pharmaceutical composition amount administered for human use allows maintaining a high therapeutically effective concentration of the target antioxidant protein in the body on a long-term basis.

The pharmaceutical composition is a source product for preparation of various dosage forms application of which is determined depending on the disease. The claimed pharmaceutical composition on the basis of the non-replicating nanoparticles with insert of the gene encoding antioxidant human proteins passed preclinical and clinical studies on study of specific (therapeutic) effectiveness and general toxic action that showed safety of this composition and therapeutic activity as antioxidant, antibacterial and detoxifying agent illustrated by further examples.

### BRIEF DESCRIPTION OF THE DRAWINGS

FIG. 1. shows evaluation of the pharmaceutical composition effect on cisplatin induced toxic reactions as compared to the control group and the experimental group.

FIG. 2. shows a pharmcacokinetic curve characterizing the blood concentration of the native human lactorferrin isolated from the donor human milk; and a pharmcacokinetic curve, characterizing blood concentration of the recombinant human lactoferrin obtained after injection of the pharmaceutical composition to mice at a dosage of 4.3×10¹¹ virus particle (v.p.) per sqm;

FIG. 3. shows a pharmacokinetic curve characterizing the blood concentration of the native human lactorferrin isolated from the donor human milk; and a pharmcacokinetic curve, characterizing blood concentration of the recombinant human lactoferrin obtained after injection of the pharmaceutical composition to mice at a dosage 4.3×10¹² virus particle (v.p.) per sqm;

FIG. 4. shows a pharmcacokinetic curve characterizing the blood concentration of the native human lactorferrin isolated from donor human milk; and a pharmcacokinetic curve, characterizing blood concentration of the recombinant human lactoferrin obtained after injection of the pharmaceutical composition to mice at a dosage of 4.3×10¹³ virus particle (v.p.) per sqm;

### EXPERIMENTAL EMBODIMENT OF INVENTION

Development of a pharmaceutical composition carrying human adenovirus 5 genome based non-replicating nanoparticles with slow expression of human lactoferrin in the human body in therapeutical qualities requires:

1) construction of non-replicating nanoparticles with the lactoferrin gene insert;

2) development of the pharmaceutical composition production process;

3) demonstration of compliance of human lactoferring expressed by non-replicating nanoparticles with native human lactoferrin;

4) determination of the path of injection, a therapeutic dosage and the allowable dosage range;

5) demonstration of the prolonged action of the pharmaceutical composition;

6) development of a safe and a therapeutically effective application of the pharmaceutical composition.

The examples, tables and figures shown further disclose the essence of the invention and confirm effectiveness of the solution by this invention.

### EXAMPLE 1

1) Construction of human adenovirus 5 genome based non-replicating nanoparticles with the human lactoferrin gene insert.

2) Obtainment of the pharmaceutical composition.

Construction of human adenovirus 5 genome non-replicating nanoparticles (dimension 70-80 nm) with the human lactoferrin gene insert was performed by a homologous recombination method in the cell culture and was conducted with use of the generally known laboratory methods (for example, Sambrook J., Frich E., Maniatis T. et al. Genetic engineering methods. Molecular cloning. M, Mir, 1984, p. 205-224, 387-420). The recombinant plasmide pJM17 (W. J. McGrory, D. S. Bautista, F. L. Graham. A simple technique for the rescue of early regionI mutations into infectious human adenovirus type 5, Virology, V, 163, No. 2, 1988, p. 614) was taken as a basis with deletion in the E1 sphere of the adenoviral genome. An artificially synthesized cDNA of human lactoferrin gene was ligated to the generally known shuttle-plasmide pRcCMV (Invitrogen, San Diego, Calif., No. V75020). Then in order to achieve reciprocal transformation of the resulting plasmide pRcCMV—Lf and the plasmide pJM17 cells 293 were transfected by them (for example, No. 300192, CLS, Germany) with the help of the Calcium-Phospate (Graham F L, van der Eb AJ, 1973—A new technique for the assay of infectivity of human adenovirus 5 DNA″.Virology, 52 (2) 456-67). As a result, non-replicating nanoparticles carrying the expressing cassette with CMV-promoter, a human lactoferrin gene and a polyadenylation signal were obtained. Recombinant particle plaques were formed on the cell culture in several days after transfection, they were taken by Pasteur pipette and the obtained material was multiplied on Line 203 cells until the titre 3×1010 v.p. (virus particle)/ml (108 pfu per ml) was achieved.

Pharmaceutical Composition Production

The required content of non-replicating nanoparticles in the composition was determined in examples 4 and 5 by antitoxic effect and must amount to no less than 2.33×1011 v.p. per ml (which corresponds to activity no less than 6.7×108 pfu per ml). The preparation volume must make 3 ml, which is related to convenience of packing and application. The composition production process is performed on several stages.

The cell suspension obtained above carrying non-replicating nanoparticles in the titre 3×1010 v.p. per ml was used for subsequent titre tailing of non-replicating nanoparticles and preparation of the finished pharmaceutical composition with the target content no less than 2.33×1011 v.p. per ml (which corresponds to activity no less than 6.7×108 pfu per ml).

A wave bioreactor with 4,500 ml of the permissive cell culture suspension 293 was seeded with a cell suspension with the volume 500 ml carrying non-replicating nanoparticles with titre 3×1010 v.p. per ml in order to increase the required non-replicating nanoparticles titre.

Non-replicating nanoparticles were cultivated inside cells until their content achieved 6×1010 v.p. per ml (activity 2×108 pfu per ml) approximately for 48 hours in order to increase the titre. On achievement of the required content of nanoparticles the cell mass was submitted for purification that included several stages:

1) Settling of the cellular mass by centrifuging. The suspension fed to the centrifuge had no less than 1014 v.p. per 51 (evaluated by mass-spectrometer, 1 OU=1012 v.p.). Centrifuged the mass in the regime 6000 g for 15 min, discharged liquid supernatant and fed the remaining solid part containing non-replicating nanoparticles for the subsequent purification stages.

2) Extracted non-replicating nanoparticles from the cell culture means of cell breakage by four-fold freezing-thawing. Prepared a buffer solution with pH 8.0: 5mMTrisHCI, 0.075 MNaCl, 1 mMMgCl2, 5% saccharose, 1% polisorbat 80. Resuspended the resulting sediment at the previous stage in 70 ml of the buffer (content ratio ×71). The solution volume amounted to 80 ml.

Froze for 2 hours in the liquid nitrogen, thawed—on the water bath (at +37° C.) preventing overheating.

3) Performed additional nuclease treatment in order to simplify subsequent removal of genome cellular DNA. Added benzoase until the concentration in the solution made 150 U/ml and set mild mixing with the help of a magnetic stirrer for 3 hours at a room temperature (21-23° C.).

4) Isolated non-replicating nanoparticles from broken cells by centrifuging at 9000 g for 10 min. Collected supernatant carrying non-replicating nanoparticles.

5) Subsequent treatment performed by ultra-filtration. Diluted the resulting supernatant by the buffer buffer OM (50 mM TrisHCl pH 7.5, 1M NaCl, 2 mM MgCl2, 5% saccharose, pH 7.5) until the volume reached no less than 200 ml, mixed by a magnetic stirrer. Permanently made up the volume of the circulating solution (a retentate) to the initial volume (200 ml).

6) Then performed purification by anion-exchange chromatography.

Applied the retentate to the column (AxiChrom 70/300 with the volume 400 ml) containing the anion-exchange sorbent Q Sepharose virus licensed. Non-replicating nanoparticles are sorbed on the column whereas admixtures are not sorbed but washed out by buffer A. After removal of admixtures desorbed non-replicating nanoparticles by buffer B washing. Chromatography conditions: flow rate 193 ml/min , buffer A (40 mM TrisHCl, 0.27 M NaCl, 2 mM MgCl 2, 5% Saccharose , 0.1% Polysorbate 80, pH 7.5), conductivity ˜28-30 mS/cm; buffer B (40 mM TrisHCl, 0.5 M NaCl, 2 mM MgCl 2, 5% saccharose, 0.1% polysorbate 80, pH 7.5) conductivity ˜50 mS/cm. Sent eluate in the volume 200 ml [symbol] to the following stage.

7) Exclusive Chromatography

Apply the eluate obtained at the previous stage on the column (AxiChrom 100/300 with the volume 800 ml), containing sorbent Q Sepharose 4 FastFlow. Eluated high-molecular substances not included in the sorbent pores by the first peak (non-replicating nanoparticles belong to them), eluated admixtures after escaping peak of non-replicating nanoparticles. Chromatography conditions: flow rate 130 ml/min, buffer (10 mMTrisHCl, 75 mMNaCl, 1 mMMgCl 2, 5% saccharose 0.05% Polysorbate 80, pH 8.0).

Add ethanol to the obtained eluate (80 ml) to concentration 0.5% and ethylenediamine tetraacetic acid (EDTA) to concentration 100 μM, sent to the following stage.

8) Normal Filtration.

Sterilization of the obtained preparation was performed by filtration through a system of filters with the pore dimension 22 μM. The final volume of the preparation at this stage amounted to 80 ml and contained non-replicating nanoparticles in the titre 1×1012 v.p. per ml. Dissolved it by the formulating buffer (for example, 10 mMTrisHCl, 75 mMNaCl, 1 mMMgCl 2, 5% saccharose, 0.05% Polysorbate 80, 0.5% ethanol, 100 μm EDTA, pH 8.0) until the target content 2.33×1011 v.p. per ml was obtained and sterilized by normal filtration.

Thus, the above-described method to obtain the pharmaceutical composition allows obtaining on the basis of the constructed non-replicating nanoparticles with the human lactoferrin gene insert the content of non-replicating nanoparticles in the product no less than 2.33×1011 v.p. per ml of the formulating buffer (which corresponds to activity of the pharmaceutical composition no less than 6.7×108 pfu per ml), and the full therapeutic dosage for the human (7×1011 v.p.) contains in 3 ml of the pharmaceutical composition, which corresponds to the set task.

### EXAMPLE 2

Comparison of native human lactoferrin and human lactoferrin expressed by non-replicating nanoparticles by physicochemical properties and biological activity.

Human adenovirus 5 genome based non-replicating nanoparticles express lactoferrin called a recombinant one, its correspondence to the native lactoferrin determined on physical and chemical properties and biological (antioxidant) activity. In order to achieve this, isolated the recombinant human lactoferrin from the permissive 293 cell culture transduced by non-replicating nanoparticles with insert of the gene encoding human lactoferrin. Conducted purification of the recombinant human lactoferrin from the cultural liquid by a standard method of affine chromatography on the matrix of activated CNBr sepharose 4B, co-valently connected with highly purified antibodies to the human lactoferrin. Analyzed physical and chemical properties by generally accepted methods of electrophoresis, Western blotting and the biological (antioxidant) activity—by a well-known method of lipid peroxygenation inhibition in the mice liver homogenate. Native lactoferrin was obtained from the donor human milk (Russian Patent No. 2165769, 13 Jul. 2000).

1) The data of the electrophoretic analysis showed that the recombinant human lactoferrin contains protein, the molecular mass of which makes 76 kDa, which corresponds to the molecular mass of the human lactoferrin extracted from the donor human milk.

2) The Western blotting method demonstrated that the recombinant protein has specific interaction with polyclonal antibodies against human lactoferrin (Sigma, Cat. No. L3262).

3) Antioxidant activity of recombinant human lactoferrin—1.3×10-6 mol/ml is comparable to activity of human milk lactoferrin -1.4×10-6 mol/ml.

Thus, the correspondence of recombinant lactoferrin expressed by human adenovirus 5 genome based non-replicating nanoparticles and of native lactoferrin from human donor milk by physical and chemical properties and antioxidant activity was established.

### EXAMPLE 3

Comparison of Antimicrobial Activity.

Correspondence of antimicrobial activity of recombinant lactoferrin as compared to native lactoferrin extracted from human milk was evaluated on the basis of reference strains and clinical isolates by a micro method in a broth with use of methodical guidance on determination of microorganism sensitivity to antibacterial medications (Methodical Guidance of 4.2.1890-04

### Determination of Microorganism Sensitivity to Antibacterial Drugs Approved by the Chief Sanitary Doctor of the RF on Mar. 4, 2004).

TABLE 1

| | Minimum growth inhibiting | | |
|---|---|---|---|
| | concentration, mg per ml | | |
| | Growth | | |
| | control | Human milk | Recombinant |
| Test-strain | medium | lactoferrin | lactoferrin |
| Candida albicans | >5 | 5 | 5-2.5 |
| CBS 8837 | | | |
| Candida parapsilosis | >5 | 1.25 | 2.5 |
| ATCC 22019 | | | |
| Staphylococcus aureus | >5 | 2.5 | 2.5 |
| ATCC 29213 | | | |
| Staphylococcus aureus | >5 | >5 | 2.5-1.25 |
| ATCC 25923 | | | |
| Staphylococcus aureus | >5 | >5 | 2.5-1.25 |
| ATCC 43300 | | | |
| Escherichia coli ATCC | >5 | >5 | 5 |
| 25922 | | | |
| Bacillus subtilus 6633 | >5 | 0.3 | 0.3 |
| Bacillus subtilus L₂ | >5 | 0.02 | 0.04 |
| Bacillus cereus | >5 | 0.6 | 0.3 |
| var mycoides HB | | | |
| Bacillus cereus 537 | >5 | 0.15 | 0.3 |
| Bacillus pumilus | >5 | 0.3 | 0.15 |
| NCTS 8241 | | | |
| Micrococcus luteus | >5 | 1.25 | 1.25 |
| ATCC 9341 | | | |
| Enterococcus faecalis | >5 | 5 | 5 |
| ATCC29212 | | | |
| Micrococcus luteus | >5 | 1.25 | 2.5 |
| ATCC 9341 | | | |
| Rhodococcus rhodochrous | >5 | 0.3 | 5 |
| ATCC 15096 | | | |

In the majority of cases the data provided in Table 1 demonstrate equal or lower minimum recombinant human lactoferrin values as compared to native human lactoferrin inhibiting standard test-microbe growth (which signals even better antimicrobial activity as compared to the native lactoferrin).

Thus, it was established that the recombinant lactoferrin expressed by non-replicating nanoparticles on the basis of human adenovirus 5 genome with the human lactoferrin gene insert and native lactoferrin extracted from human milk have similar antimicrobial activity.

### EXAMPLE 4

Evaluation of detoxifying action with determination of the minimum effective dosage.

The detoxifying action of the pharmaceutical composition was evaluated in vivo with use of laboratory animals on the toxicosis model induced by the cytostatic drug cisplatin widely used in polychemotherapy schemes of patients affected by malignant processes.

The antineoplastic action of cisplatin is realized due to formation of free radical products that damage DNA by intra- and interstrand DNA cross-links and induce lipid peroxygenation of cellular membranes of liver, lungs and other organs. This finally results in disturbance of functions of many organs and induces a large complex of toxic reactions, the intensity of which increases with increase of the cisplatin dosage.

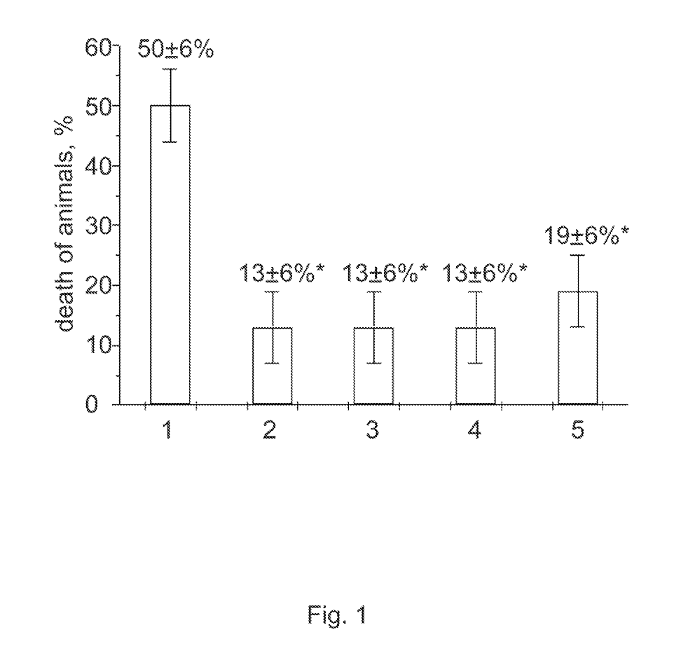

In order to evaluate effect of the pharmaceutical composition on cisplastin-induced toxic reaction it was injected at a dosage of 16 mg per kg, which resulted in loss of control animals in 50±6% of cases from acute toxic reactions (FIG. 1). The animal loss started from Day 4 after the exposure and continued for 5 days. Toxic reactions were expressed in increase of AST and ALT-enzyme activity increase, elevation of creatinine level on Day 7 and of urea on Day 3 and 7 of observation (Table 2).

Non-replicating nanoparticles with the human lactoferrin gene insert at dosages 1.4×10¹¹, 4.3×10¹¹, 4.3×10¹² v.p. per sqm as the detoxifying agent were administered as a single injection to experimental animal in 72 hours before intravenous cisplatin injection and the control animals received a triple injection of native human lactoferrin from donor human milk at a single dosage 10 mg per kg (a course dosage 30 mg/kg), the first injection in 24 hours after cisplatina infusion.

FIG. 1 represents data where:

1—loss of control animals, cisplatin was injected at a single dosage 16 mg/kg intravenously;

2—loss of experimental animals that received a pharmaceutical composition at a single dosage of 1.4×10¹¹ v.p. per sqm in 72 hrs before cisplatin injection;

3—loss of experimental animals that received a pharmaceutical composition at a single dosage of 4.3×10¹¹ v.p. per sqm in 72 hrs before cisplatin injection;

4—loss of experimental animals that received a pharmaceutical composition at a single dosage of 4.3×10¹² v.p. per sqm in 72 hrs before cisplatin injection;

5—control animals that received native human lactoferrin from donor human milk in 24 hrs cisplatin injection at a single dosage 10 mg/kg for subsequent 3 days (a course dosage 30 mg/kg).

Thus, in the period of the cisplatin toxic action the maximum production of recombinant human lactoferrin was observed in experimental animals that remained unchanged for 10 days inducing reduction of animal loss up to 13±6% following injection of non-replicating nanoparticles with the human lactoferrin gene insert at dosages 1.4×10¹¹, 4.3×10¹¹ and 4.3×10¹² v.p. per sqm. The lethality rate in the control group where animals received three injections of native human lactoferrin reached 19±6%.

The obtained results signify detoxifying effect of the pharmaceutical composition producing recombinant lactoferrin and of native lactoferrin extracted from donor human milk against the toxic cisplatin action.

Biochemical study results confirmed reduction of cisplatin toxic reaction intensity following injection of the pharmaceutical composition at dosages 4.3×10¹¹ v.p. per sqm and 4.3×10¹² v.p. per sqm.

TABLE 2

| | Biochemical blood parameters of animals | | |
|---|---|---|---|
| Effect | Day 4 | Day 7 | Day 10 |

AST, MU per 1

| | | | |
|---|---|---|---|
| Cisplatin | 201 ± 15 | 289 ± 15 | 266 ± 21 |
| Pharmaceutical composition (4.3 × | 174 ± 10 | 167 ± 17 | 140 ± 10 |
| 10¹¹ ) + cisplatin | | | |
| Pharmaceutical composition (4.3 × 10¹²) + | 178 ± 8 | 187 ± 23 | 123 ± 12 |
| cisplatin | | | |
| cisplatin + native human lactoferrin | 171 ± 13 | 156 ± 17 | 156 ± 10 |

| | |
|---|---|
| Physiological solution | 123 ± 25 |

ALT, MU per 1

| | | | |
|---|---|---|---|
| Cisplatin | 79 ± 10 | 125 ± 8 | 61 ± 3 |
| Pharmaceutical composition (4.3 × | 56 ± 14 | 68 ± 13 | 64 ± 9 |
| 10¹¹) + cisplatin | | | |
| Pharmaceutical composition (4.3 × | 61 ± 1 | 69 ± 9 | 60 ± 10 |
| 10¹²) + cisplatin | | | |
| cisplatin + native human lactoferrin | 45 ± 8 | 75 ± 15 | 51 ± 8 |
| Physiological solution | 48 ± 12 | | |

Creatinine, μmol per 1

| | | | |
|---|---|---|---|
| Cisplatin | 65 ± 12 | 97 ± 10 | 124 ± 21 |
| Pharmaceutical composition (4.3 × | 51 ± 11 | 54 ± 13 | 59 ± 13 |
| 10¹¹) + cisplatin | | | |
| Pharmaceutical composition (4.3 × | 46 ± 9 | 67 ± 16 | 66 ± 11 |
| 10¹²) + cisplatin | | | |
| cisplatin + native human lactoferrin | 46 ± 5 | 57 ± 15 | 56 ± 6 |
| Physiological solution | 35 ± 9 | | |

Urea, μmol per 1

| | | | |
|---|---|---|---|
| Cisplatin | 10.5 ± 0.9 | 11.0 ± 1.1 | 6.1 ± 0.9 |
| Pharmaceutical composition (4.3 × | 5.4 ± 1.8 | 5.9 ± 1.2 | 4.8 ± 0.5 |
| 10¹¹) + cisplatin | | | |
| Pharmaceutical composition (4.3 × | 6.0 ± 0.8 | 5.4 ± 0.9 | 6.2 ± 1.5 |
| 10¹²) + cisplatin | | | |
| cisplatin + native human lactoferrin | 5.4 ± 0.3 | 6.0 ± 0.5 | 5.2 ± 0.5 |
| Physiological solution | 4.7 ± 1.0 | | |

Thus, Table 2 represents data that show that practically all biochemical blood value parameters with cisplatin injection on the background of the pharmaceutical composition and of native protein effect corresponded to average statistical normal values during all periods of observation. Values of the liver functional state—AST and ALT didn't increase in these groups to such a great extent that in control animals and values reflecting the functional kidney state—creatinine and urea—retained values close to normal as opposed to significantly increased values in control groups, i.e. the recombinant human lactoferrin as well as the native one extracted from human milk, contributes to reduction of cytostatic-induced nephro- and hepatotoxicity.

Thus, the recombinant human lactoferrin has detoxifying action with respect to cisplatin toxicity, which demonstrates its functional activity, a similar one to the native protein.

Evaluation of the detoxifying effect of the pharmaceutical composition on chemical-induced toxicosis models allowed determining the minimum therapeutically effective dosage equal to 4.3×10¹¹ v.p. per sqm (a toxic dosage (TD) is specified as the non-replicating nanoparticles per sq m of the animal body surface, dosage of drugs calculated per the body surface for various types of animals and a man are equivalent. (Khabriev R. U. Guidance on experimental (preclinical) study of new pharmacological substances, 2000, p. 98).

### EXAMPLE 5

Determination of tolerable dosage thresholds

Values of tolerable dosage thresholds of the pharmaceutical composition were determined in experiments following a single intravenous injection (“acute” toxicity) to laboratory animals at dosages: 4.3×1011 v.p. per sqm, 43.0×1011 v.p. per sqm, 215.0×1011 v.p. per sqm, 430.0×1011 v.p. per sqm and 860.0×1011 v.p. per sqm.

In acute toxicity experiments the minimum therapeutic dosage calculated per sqm was increased by 10, 50, 100 and 200 times. TD increase by 10-200 times is sufficient for receipt of reliable information about toxicological safety of the studied pharmacological product. The minimum effective dosage equal to 4.3×1011 v.p. per sqm and the way of injection were chosen on the basis of study results obtained in the course of its pharmacological activity investigation on animals with grave exogenous intoxication caused penetration in animals of toxic and lethal dosages of highly toxic antineoplastic drug (example 4).

The following data were registered for the experimental animals after single intravenous injection of the pharmaceutical composition in the above-stated dosages: clinical signs of possible intoxication, toxicity-induced loss, day of loss, change of body weight. Weighing of animals was performed before the drug injection (background) and on Day 3, 7, 10, 14, 21 and 30 after the drug injection. On Day 30 all survived animals were euthanized with subsequent autopsy. The postmortem examination of all animals was performed including macroscopic evaluation of the state of body cavities, internal organs and tissues. The local irritating action of the medication was evaluated visually on examination of the injection site. Control animals received the stabilizing buffer solution (control substance No. 1) and isotonic (0.9%) solution of potassium chloride (control substance No. 2).

The obtained data characterizing toxicity of the pharmaceutical composition after a single intravenous application by mice are represented in Tables 3, 4, 5.

TABLE 3

| | | | | Toxicity induced | | |
|---|---|---|---|---|---|---|
| | | | | animal loss | | |
| | | | Total | | | Day |
| | Experimental | Dosage, | number | | | of |
| Group | or control | v.p. | of | Total | | loss, |
| No. | substance | per sqm | animals | loss | % | day |

Males

| | | | | | | |
|---|---|---|---|---|---|---|
| 1 | Pharmaceutical | 4.3 × 10¹¹ | 6 | 0 | 0 | — |
| | composition | | | | | |
| 2 | Pharmaceutical | 43.0 × 10¹¹ | 6 | 0 | 0 | — |
| | composition | | | | | |
| 3 | Pharmaceutical | 215.0 × 10¹¹ | 6 | 0 | 0 | — |
| | composition | | | | | |
| 4 | Pharmaceutical | 430.0 × 10¹¹ | 6 | 0 | 0 | — |
| | composition | | | | | |
| 5 | Pharmaceutical | 860.0 × 10¹¹ | 6 | 2 | 33 | 2 and |
| | composition | | | | | 9 |
| 6 | Stabilizing buffer | 25 ml/kg | 6 | 0 | 0 | — |
| | (control substance | | | | | |
| | No. 1) | | | | | |
| 7 | Stabilizing buffer | 50 ml/kg | 6 | 0 | 0 | — |
| | (control substance | | | | | |
| | No. 1) | | | | | |
| 8 | NaCl solution | 50 ml/kg | 6 | 0 | 0 | — |
| | 0.9% (control | | | | | |
| | substance No. 2) | | | | | |

Females

| | | | | | | |
|---|---|---|---|---|---|---|
| 1 | Pharmaceutical | 4.3 × 10¹¹ | 6 | 0 | 0 | — |
| | composition | | | | | |
| 2 | Pharmaceutical | 43.0 × 10¹¹ | 6 | 0 | 0 | — |
| | composition | | | | | |
| 3 | Pharmaceutical | 215.0 × 10¹¹ | 6 | 0 | 0 | — |
| | composition | | | | | |
| 4 | Pharmaceutical | 430.0 × 10¹¹ | 6 | 0 | 0 | — |
| | composition | | | | | |
| 5 | Pharmaceutical | 860.0 × 10¹¹ | 6 | 1 | 17 | 13 |
| | composition | | | | | |
| 6 | Stabilizing buffer | 25 ml/kg | 6 | 0 | 0 | — |
| | (control substance | | | | | |
| | No. 1) | | | | | |
| 7 | Stabilizing buffer | 50 ml/kg | 6 | 0 | 0 | — |
| | (control substance | | | | | |
| | No. 1) | | | | | |
| 8 | 0.9% NaCl solution | 50 ml/kg | 6 | 0 | 0 | — |
| | (control substance | | | | | |
| | No. 2) | | | | | |

The experimental results shown in Table 3 demonstrate that a single intravenous injection of the pharmaceutical composition to male and female mice at dosages from 4.3×1011 v.p. per sqm to 430×1011 v.p. per sqm was well tolerated by animals. No toxicity induced loss was registered. Administration of the pharmaceutical composition at a dosage equal to 860×1011 v.p. per sqm induced mice loss from toxic action. The loss rate in the male mice group amounted to 33%, and in the female mice group—17%. The average loss of mice (female and male) with use of this drug dosage amounted to 25%. There were no intoxications signs on lost animals.

TABLE 4

| | | | |
|---|---|---|---|
| | | Dosage, | |
| Group | Experimental or | v.p. | |
| No. | control substance | per sqm | External intoxication signs |

Males

| | | | |
|---|---|---|---|
| 1 | Pharmaceutical | 4.3 × 10¹¹ | Short-term hypodynamia |
| | composition | | lasting 3 hours |
| 2 | Pharmaceutical | 43.0 × 10¹¹ | Short-term hypodynamia |
| | composition | | lasting 3 hours |
| 3 | Pharmaceutical | 215.0 × 10¹¹ | Short-term hypodynamia |
| | composition | | lasting 3 hours |
| 4 | Pharmaceutical | 430.0 × 10¹¹ | Short-term hypodynamia |
| | composition | | lasting 3 hours |
| 5 | Pharmaceutical | 860.0 × 10¹¹ | Short-term hypodynamia |
| | composition | | lasting 3 hours |
| 6 | Stabilizing buffer | 25 ml/kg | No external intoxication signs |
| | (control substance | | |
| | No. 1) | | |
| 7 | Stabilizing buffer | 50 ml/kg | No external intoxication signs |
| | (control substance | | |
| | No. 1) | | |
| 8 | 0.9% NaCl | 50 ml/kg | No external intoxication signs |
| | solution (control | | |
| | substance No. 2) | | |

Females

| | | | |
|---|---|---|---|
| 1 | Pharmaceutical | 4.3 × 10¹¹ | Short-term hypodynamia |
| | composition | | lasting 3 hours |
| 2 | Pharmaceutical | 43.0 × 10¹¹ | Short-term hypodynamia |
| | composition | | lasting 3 hours |
| 3 | Pharmaceutical | 215.0 × 10¹¹ | Short-term hypodynamia |
| | composition | | lasting 3 hours |
| 4 | Pharmaceutical | 430.0 × 10¹¹ | Short-term hypodynamia |
| | composition | | lasting 3 hours |
| 5 | Pharmaceutical | 860.0 × 10¹¹ | Short-term hypodynamia |
| | composition | | lasting 3 hours |
| 6 | Stabilizing buffer | 25 ml/kg | No external intoxication signs |
| | (control substance | | |
| | No. 1) | | |
| 7 | Stabilizing buffer | 50 ml/kg | No external intoxication signs |
| | (control substance | | |
| | No. 1) | | |
| 8 | NaCl solution | 50 ml/kg | No external intoxication signs |
| | 0.9%(control | | |
| | substance No. 2) | | |

Table 4 shows data on intoxication—after injection of the pharmaceutical composition at all studied dosages in mice (in male and female animals) external intoxication signs were observed as a long-term (lasting 1-3 hours) reduction of motor activity (hypodynamia). In 24 hours following injection and further during the whole period of animal observation (30 days) no external intoxication signs were observed. Animals were active, the reaction for the man, tactile and pain irritant response was intense.

TABLE 5

| | Experimental | | | | | | | | |
|---|---|---|---|---|---|---|---|---|---|
| | or | Dosage, | Animal body weight, g | | | | | | |
| | control | v.p. | | Day following injection | | | | | |
| Group No. | substance | per sqm | Background | 3 | 7 | 10 | 14 | 21 | 30 |

Males

| | | | | | | | | | |
|---|---|---|---|---|---|---|---|---|---|
| 1 | Pharmaceutical | 4.3 × 10¹¹ | 20.7 ± 0.2 | 20.9 ± 0.1 | 21.3 ± 0.2 | 22.6 ± 0.2 | 23.2 ± 0.3 | 24.6 ± 0.3 | 26.0 ± 0.3 |
| | composition | | | | | | | | |
| 2 | Pharmaceutical | 43.0 × 10¹¹ | 20.9 ± 0.1 | 21.1 ± 0.2 | 22.0 ± 0.1 | 23.0 ± 0.1 | 23.9 ± 0.2 | 25.4 ± 0.3 | 26.5 ± 0.3 |
| | composition | | | | | | | | |
| 3 | Pharmaceutical | 215.0 × 10¹¹ | 21.0 ± 0.1 | 21.4 ± 0.2 | 22.1 ± 0.2 | 23.2 ± 0.2 | 23.9 ± 0.1 | 25.1 ± 0.2 | 26.4 ± 0.3 |
| | composition | | | | | | | | |
| 4 | Pharmaceutical | 430.0 × 10¹¹ | 20.8 ± 0.2 | 21.7 ± 0.2 | 22.7 ± 0.1 | 23.1 ± 0.2 | 23.8 ± 0.4 | 25.1 ± 0.4 | 26.1 ± 0.5 |
| | composition | | | | | | | | |
| 5 | Pharmaceutical | 860.0 × 10¹¹ | 21.3 ± 0.1 | 21.9 ± 0.7 | 21.7 ± 0.8 | 23.2 ± 0.1 | 24.0 ± 0.2 | 26.5 ± 0.2 | 26.9 ± 0.2 |
| | composition | | | | | | | | |
| 6 | Stabilizing | 25 ml/kg | 20.6 ± 0.1 | 21.2 ± 0.2 | 21.9 ± 0.2 | 22.7 ± 0.3 | 23.6 ± 0.3 | 25.2 ± 0.2 | 26.6 ± 0.2 |
| | buffer | | | | | | | | |
| | (control | | | | | | | | |
| | substance | | | | | | | | |
| | No. 1) | | | | | | | | |
| 7 | Stabilizing | 50 ml/kg | 21.1 ± 0.1 | 22.0 ± 0.2 | 22.8 ± 0.2 | 23.7 ± 0.3 | 25.0 ± 0.3 | 26.2 ± 0.2 | 27.4 ± 0.3 |
| | buffer | | | | | | | | |
| | (control | | | | | | | | |
| | substance | | | | | | | | |
| | No. 1) | | | | | | | | |
| 8 | NaCl | 50 ml/kg | 21.5 ± 0.3 | 23.0 ± 0.7 | 24.5 ± 0.7 | 25.0 ± 0.6 | 25.9 ± 0.9 | 26.5 ± 0.7 | 27.4 ± 0.7 |
| | solution | | | | | | | | |
| | 0.9% (control | | | | | | | | |
| | substance | | | | | | | | |
| | No. 2) | | | | | | | | |

Females

| | | | | | | | | | |
|---|---|---|---|---|---|---|---|---|---|
| 1 | Pharmaceutical | 4.3 × 10¹¹ | 21.3 ± 0.2 | 21.6 ± 0.2 | 22.0 ± 0.1 | 22.7 ± 0.1 | 23.3 ± 0.1 | 23.8 ± 0.1 | 24.4 ± 0.4 |
| | composition | | | | | | | | |
| 2 | Pharmaceutical | 43.0 × 10¹¹ | 20.9 ± 0.1 | 21.1 ± 0.1 | 21.7 ± 0.1 | 21.9 ± 0.3 | 22.3 ± 0.2 | 22.7 ± 0.1 | 24.4 ± 0.2 |
| | composition | | | | | | | | |
| 3 | Pharmaceutical | 215.0 × 10¹¹ | 21.5 ± 0.2 | 21.8 ± 0.1 | 22.3 ± 0.1 | 22.4 ± 0.2 | 22.6 ± 0.1 | 23.1 ± 0.1 | 24.6 ± 0.2 |
| | composition | | | | | | | | |
| 4 | Pharmaceutical | 430.0 × 10¹¹ | 21.6 ± 0.2 | 21.2 ± 0.3 | 22.8 ± 0.2 | 23.0 ± 0.2 | 23.2 ± 0.2 | 24.7 ± 0.3 | 25.4 ± 0.2 |
| | composition | | | | | | | | |
| 5 | Pharmaceutical | 860.0 × 10¹¹ | 21.4 ± 0.3 | 20.5 ± 0.4 | 21.6 ± 0.5 | 21.9 ± 0.6 | 23.0 ± 0.8 | 24.9 ± 0.5 | 25.4 ± 0.5 |
| | composition | | | | | | | | |
| 6 | Stabilizing | 25 ml/kg | 20.7 ± 0.1 | 20.9 ± 0.1 | 21.2 ± 0.1 | 21.4 ± 0.3 | 22.1 ± 0.1 | 22.7 ± 0.3 | 24.2 ± 0.3 |
| | buffer | | | | | | | | |
| | (control | | | | | | | | |
| | substance | | | | | | | | |
| | No. 1) | | | | | | | | |
| 7 | Stabilizing | 50 ml/kg | 20.5 ± 0.1 | 20.7 ± 0.1 | 21.1 ± 0.1 | 21.5 ± 0.1 | 21.8 ± 0.1 | 22.3 ± 0.2 | 24.3 ± 0.3 |
| | buffer | | | | | | | | |
| | (control | | | | | | | | |
| | substance | | | | | | | | |
| | No. 1) | | | | | | | | |
| 8 | NaCl | 50 ml/kg | 18.3 ± 0.1 | 19.0 ± 0.2 | 20.1 ± 0.3 | 20.1 ± 0.2 | 20.0 ± 0.2 | 22.0 ± 0.4 | 23.8 ± 0.3 |
| | solution | | | | | | | | |
| | 0.9% (control | | | | | | | | |
| | substance | | | | | | | | |
| | No. 2) | | | | | | | | |

The data shown in Table 5 demonstrate that the body weight of mice that received the pharmaceutical composition at single intravenous dosages from 4.3×1011 v.p. per sqm to 860,0×1011 v.p. per sqm, as well as the body weight of control animals that received the stabilizing buffer solution and the isotonic (0.9%) solution of potassium chloride (of males as well as females) was uniformly increased during the whole period of observation of animals (30 days).

Autopsy of loss mice demonstrated venous hyperemia of internal organs and namely, of spleen and liver. No macroscopically pathological changes related to the composition action in other organs and tissues of mice were not identified.

As a result of conducted studies the maximum tolerable and lethal dosage of the pharmaceutical composition for mice following its single intravenous injection were determined:

dosage 430.0×1011 v.p. per sqm is characterized as the maximum tolerable dosage;

dosage 860.0×1011 v.p. per sqm is characterized as the partially lethal dosage inducing loss of 25% of animals.

No differences in sensitivity of female and male animals accepted for the animal study to toxic action of the pharmaceutical composition after its single intravenous application were found.

External signs of intoxication in mice after use of tolerable and maximum tolerable use were represented by hypodynamia or adynamia, the extent of intensity and duration of which increased with increase of the pharmaceutical composition dosage.

Use of the lethal dosage of pharmaceutical composition in mice (860.0×10¹¹ v.p. per sqm) resulted in loss of animals on Day 2-13 following injection without clearly marked clinical intoxication signs. Autopsy of lost animals and accurate postmortem examination didn't allow determining the reason of mice death.

Thus, it was concluded by experimental results that a single intravenous injection of the pharmaceutical composition to mice at dosages from 4.3×1011 v.p. per sqm to 430×1011 v.p. per sqm is satisfactorily tolerated and might be recommended within the specified range for human intravenous injection.

### EXAMPLE 6

Determination of Action Prolongation

Pharmacokinetics study is one of the major aspects of preclinial study of medications as it allows developing application regimes in clinical conditions.

The pharmacokinetics of the pharmaceutical composition was evaluated by expression of the recombinant lactoferrin by the ELISA assay with determination of the standard pharmacokinetic parameters: the maximum blood serum concentration of the target protein (Cmax), time to peak blood serum concentration (tmax), area under the pharmacokinetic curve Concentration—time (AUC) and elimination half-life period (t1/2). The pharmacokinetics was studied after the single intravenous injection of the pharmaceutical composition at dosages 4.3×1011, 4.3×1012 and 4.3×1013 v.p. per sqm. Dosages were selected on the maximum tolerated dosages (MTD) of the pharmaceutical composition determined in experiments on study of its acute toxicity on mice where MTD for mice is 4.3×1013 v.p. per sqm, after injection of which the maximum production of the recombinant human lactoferrin in the blood serum of experimental animals was observed.

Human lactoferrin extracted from human milk that was injected at a single dosage 10 mg/kg was used as a control substance.

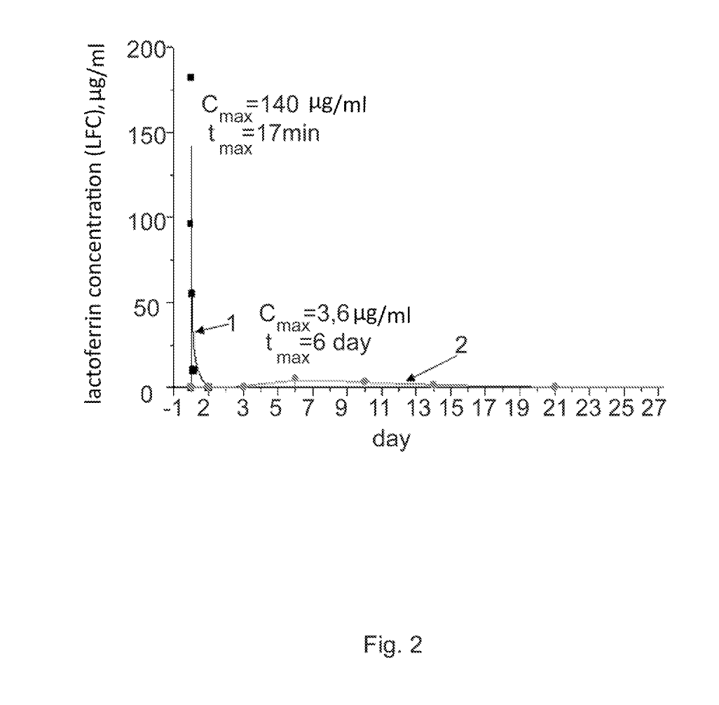

FIG. 2 shows:

a pharmcacokinetic curve 1 (▪) that characterizes blood concentration of native human lactoferrin from donor human milk;

a pharmcacokinetic curve 2 () that characterizes blood concentration of recombinant human lactoferrin registered after injection of the pharmaceutical composition to mice at a dosage of 4.3×1011 v.p. per sqm;

Cmax—maximum concentration lactoferrin in the blood serum;

tmax—time to peak lactoferrin concentration in the blood serum.

Thus, it was shown that the concentration of native human lactoferrin (curve 1) reached the maximum level −140±32 μkg/m1 in 17 min (0.011 days) after injection, the elimination half-period reached t1/2 2 hours. Following single injection of the pharmaceutical composition at a dosage of 4.3×1011 v.p. per sqm Cmax of human lactoferrin (curve 2) made 3.6±0.5 μkg/m1 and achieved the maximum level on Day 6. This protein concentration was maintained in the blood serum for 3 days, which demonstrates the dynamic balance between the target protein production and its elimination. The two curves have different form, different maximum value and the unequal time to peak ration, but the area under curve are close in values and, consequently, both drugs provide entry in the blood of the equal amounts of the drug. The elimination half-period (t1/2) of human lactoferrin following the pharmaceutical composition injection makes 8.4 days, which exceeds in 105 times the one after injection of native lactoferrin (t1/2=0.08 days) and correspondingly confirms increase of time of the human lactoferrin presence in the blood flow.

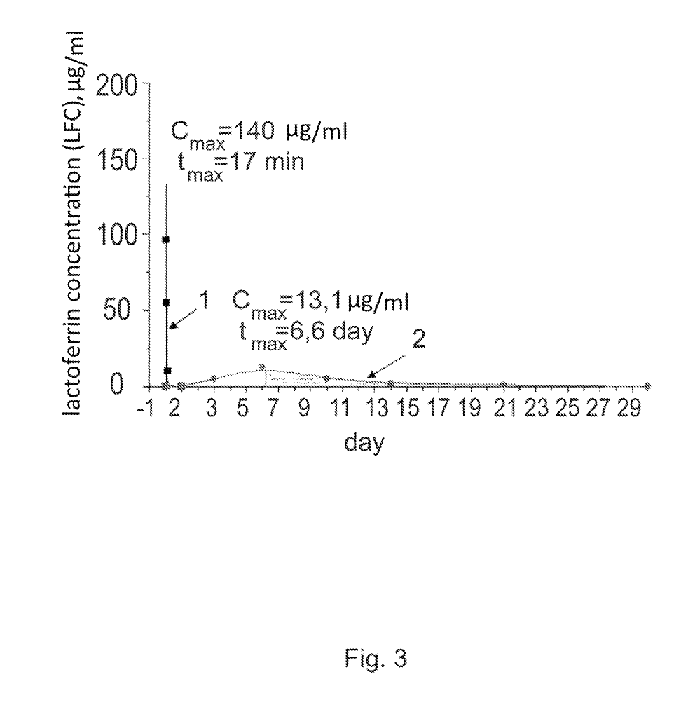

FIG. 3 shows:

a pharmcacokinetic curve 1 (▪) that characterizes blood concentration of native human lactoferrin from donor human milk;

a pharmcacokinetic curve 2 ()—that characterizes blood concentration of recombinant human lactoferrin registered after injection of the pharmaceutical composition to mice at a dosage of 4.3×1012 v.p. per sqm;

Cmax—maximum concentration lactoferrin in the blood serum;

tmax—time to peak lactoferrin concentration in the blood serum.

Following the intravenous injection of the pharmaceutical composition at a dosage of 4.3×1012 v.p. per sqm Cmax increases by approximately 4 times compared to injection of the pharmaceutical composition at a dosage of 4.3×1011 v.p. per sqm and in spite of the fact that it remains lower than after injection of native lactoferrin the area under curve 2 (Pharmaceutical composition) in by 5 times greater than the area under curve 1 due to permanent production of the recombinant protein during the long period of time.

Injection of the pharmaceutical composition at a dosage of 4.3×1013 v.p. per sqm allows reliable evaluation of non-replicating nanoparticles entry into organs (by the target protein production) and elimination from them. That's why the pharmacokinetics was evaluated following injection of the pharmaceutical composition at a dosage of 4.3×1013 v.p. per sqm as well.

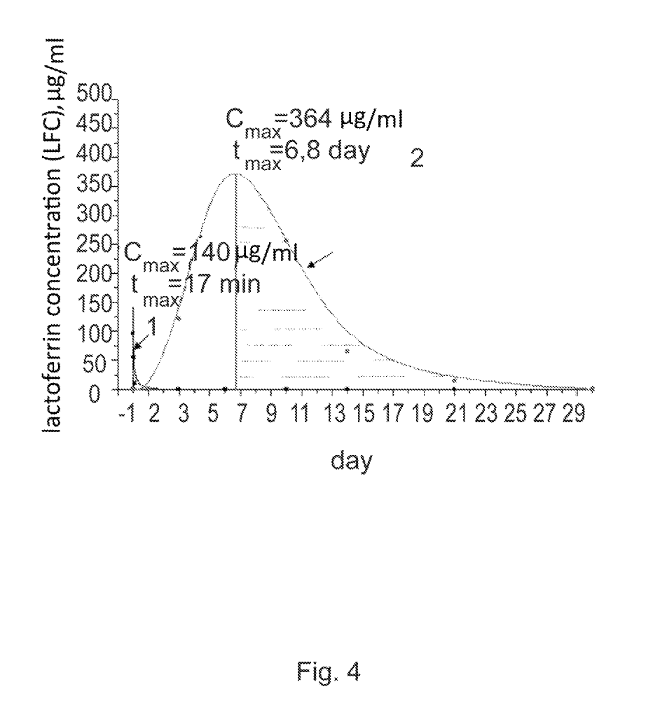

FIG. 4 shows:

a pharmcacokinetic curve 1 (▪) that characterizes blood concentration of native human lactoferrin from donor human milk;

a pharmcacokinetic curve 2 () that characterizes blood concentration of recombinant human lactoferrin registered after injection of the pharmaceutical composition to mice at a dosage of 4.3×1011 v.p. per sqm;

Cmax—maximum concentration lactoferrin in the blood serum;

tmax—time to peak lactoferrin concentration in the blood serum.

Following intravenous injection of the pharmaceutical composition at a dosage of 4.3×1013 v.p. per sqm in 6.8 days the maximum concentration of recombinant human lactoferrin was achieved equal to 364 μkg/m1 with subsequent two-phase reduction of the produced human lactoferrin in the blood serum. The figure demonstrates that the first phase of biological distribution lasted for 9.4 hours and the elimination half-period (t1/2) made 5.4. days, the second phase −13.8 days with t1/2=4.8 days. The area under the pharmacokinetic curve 2 (Pharmaceutical composition) is by 73 times more than under curve 1.

Thus, comparison of the total concentration of the recombinant human lactoferrin present in the animal bodies following injection of various dosages of the pharmaceutical composition allows making the conclusion that the dosage 4.3×1011 v.p. per sqm produces recombinant human lactoferrin approximately in the amount corresponding to the single intravenous injection of native lactoferrin at a dosage of 10 mg/kg, the excess of the composition dosage in 10 times (4.3×1012 v.p. per sqm) leads to increase of the lactoferring production by 3.6 times and injection of the pharmaceutical composition at a dosage of 4.3×1013 v.p. per sqm—by 101 times. Pharmacokinetic curves reflect prolonged action of the pharmaceutical composition from 12 hours to 30 days after the injection.

### EXAMPLE 7

Determination of the pharmaceutical composition administration way.

Evaluation of the local irritating action of the pharmaceutical composition after its single intravenous injection was performed macroscopically and with use of microscopic (histological) study methods.

The study was conducted on 12 rabbits of Chinchilla species, on males. The pharmaceutical composition was injected to rabbits intravenously (to the marginal vein of the ear) in the volume of 1.0 ml at a dosage of 4.3×1011 v.p. per sqm. Control animals were intravenously injected with the isotonic (0.9%) solution of sodium chloride in the same regime.

A material (a fragment of the rabbit ear with the marginal vein) for the microscopic (histological) study was collected on Day 3 and 14 after injection of the pharmaceutical composition.

Fragmets of the ear with the vein was fixed in 10% neutral formalin, after which 2 transversely cut ear parts with the vein were taken from each sample and that were subjected to subsequent generally accepted histological treatment including washing in flow water, water removal in alcohols, impregnation in chloroform and paraffin, paraffin embedding, cutting of paraffin blocks on a microtome. Sections with the thickness 5 μm after deparaffinization were stained by hematoxylin and eosin, included in Canada turpentine. Histological preparations were studied in the light microscope of series MS 300 by Micros company (Austria) with amplifications 100, 400, 1000.

Criteria of the local irritating action evaluation included:

macroscopic changes: external changes on the part of the vessel (vessel induration, hyperema of the surrounding skin and an inflammatory reaction in the vessel path area);

microscopic criteria: pathological changes of the vein walls, thrombophlebitis development, clotting.

Visual (macroscopic) analysis of the pharmaceutical composition injection site showed that no following signs were observed in rabbits in the site of the medication injection (marginal vein of the ear) during the whole observation period: vessel induration, hyperema of the surrounding skin and an inflammatory reaction in the vessel path area. Thus, no macroscopic signs demonstrating the local irritating action of the pharmaceutical composition following its single intravenous injection were found.

The histological study of the pharmaceutical composition injection site the following picture was observed:

1) On Day 3 and 14 following intravenous injection of 0.9% sodium chloride solution the vein lumen of all control rabbits was enlarged, free, and with some content of blood. No pathological changes of the vein walls and surrounding subdermal tissues were identified.

2) On Day 4 after the single intravenous injection of the pharmaceutical composition veins of all rabbits that received the pharmaceutical composition were enlarged with some blood content. Some bulging of the skin over the vessel was observed in all animals. The histological study didn't show any pathological changes of the vein wall and surrounding tissues in 2 rabbits. A small edema was observed in one rabbit—release of plasma to the vein wall and the surrounding subcutaneous tissue.

On Day 14 after the intravenous injection of the pharmaceutical composition the vein lumen of all rabbits was not enlarged and contained a small amount of blood. No signs of edema or other pathological changes in the vein wall or surrounding tissues were found.

Thus, a single intravenous injection of the pharmaceutical composition rendered a mild and completely reversible local irritating action. No contraindications for intravenous application of the pharmaceutical composition were found.

### EXAMPLE 8

Determination of the necessity to dilute the product for implementation of the way of administration.

Due to the fact that in the course of the previous studies (example 4) an intravenous way of administration of the pharmaceutical composition was recommended, the experimental evaluation of its compatibility with blood was performed. The hemolytical potential of the pharmaceutical composition and the formulating buffer was assessed in this way.

The pharmaceutical composition and the formulating buffer were diluted by 0.9% sodium chloride solution. The samples were incubated in the thermostat at a temperature of 370C.

TABLE 6

| | Hematocytolysis in % | | | | | | | |
|---|---|---|---|---|---|---|---|---|
| Incubation | Pharmaceutical composition | | | | | | | |
| time | | Diluted | Diluted | | | | | |
| of | Without | by 80 | by 800 | Formulating buffer | | | | |
| tested | dilution, | times, | times, | | Diluted | Diluted | Sodium | |
| substances | 2.33 × 1011 | 2.9 × 108 | 2.9 × 107 | | by | by | chloride | |
| with | v.p. per | v.p. per | v.p. per | Without | 80 | 800 | 0.9% | 0.1% |
| blood | ml | ml | ml | dilution | times, | times, | solution | solution |
| 30 min | 2.8 ± 0.7 | 1.3 ± 0.1 | 1.1 ± 0.2 | 1.7 ± 0.6 | 1.5 ± 0.1 | 1.9 ± 0.3 | 2.1 ± 0.4 | 100 |
| 1 hour | 3.4 ± 0.9 | 1.4 ± 0.1 | 1.5 ± 0.1 | 3.9 ± 0.4 | 2.2 ± 0.7 | 2.0 ± 0.5 | 2.2 ± 0.2 | |
| 2 hours | 4.5 ± 0.6 | 1.6 ± 0.3 | 1.7 ± 0.6 | 5.8 ± 0.7 | 2.3 ± 0.4 | 1.9 ± 0.1 | 1.6 ± 0.4 | |
| 3 hours | 25.9 ± 4.9 | 2.1 ± 0.3 | 1.9 ± 0.3 | 36.0 ± 0.7 | 2.8 ± 0.1 | 2.8 ± 0.1 | 1.8 ± 0.4 | |
| 4 hours | 94.5 ± 2.5 | 2.6 ± 0.1 | 2.4 ± 0.1 | 84.5 ± 6.5 | 2.3 ± 0.1 | 2.6 ± 0.2 | 2.8 ± 0.4 | |
| 24 hours | 100 | 1.9 ± 0.1 | 1.5 ± 0.2 | 100 | 2.8 ± 0.2 | 2.5 ± 0.3 | 2.5 ± 0.4 | |

The data provided in Table 6 show that during the 1st hour there was no hematocytolysis (the percent of the hematocytolysis in experimental and control (blood incubation with 0.9% sodium chloride solution) samples amounted to 3.4±0.9% and 2.2±0.2%, respectively). Subsequent incubation from 2 to 4 hours induced hematocytolysis in experimental samples didn't exceed 2.8%. Thus, injection of the non-diluted formulating buffer starting from 30 minutes after the exposure the time of complete hematocytolysis occurred in both cases in 24 hours. Hematocytolysis in the control substance (0.9% sodium chloride solution). Analogous results were obtained on evaluation of the hemolytic potential of the stabilizing buffer solution. Thus, incubation of the buffer solution with blood for 1 hour resulted in absence of hematocytolysis in experimental samples and increase of the incubation time from 2 to 24 hours induced intensification of hematocytolysis in samples from 5.8±0.7% to 100%.

Thus, the data obtained in the model system demonstrate the presence of the hemolytic activity in the pharmaceutical composition. The hemolytic activity of the pharmaceutical composition is preconditioned by the hemolytic activity of the formulation buffer.

Thus, the conclusion was made that the 80- and 800-fold dilution of the 0.9% solution of sodium chloride allowed reduction of the hemolytic activity of the medication to control values (calculated on the basis of 0.9% sodium chloride solution).

### EXAMPLE 9

Determination of Acceptable Solvent

Studied solutions acceptable for intravenous injection—water for injections, 5% glucose solution, 10% glucose solution, 0.9% sodium chloride solution—for intravenous injection of the pharmaceutical composition in 80-fold dilution (example 8) for a man. Evaluated stability of non-replicating nanoparticles in the pharmaceutical composition by dilution by any of this solutions.

Diluted 3 ml of the composition with content 2.33×1011 v.p. per ml in 200 ml of the solvent, obtained 80-fold dilution, which corresponded to 2.9×108 v.p. per ml in order to conduct the experiment. Applied the mixture after a certain time of exposure at a certain temperature the mixture on the permissive cell culture, incubated at 370C and 5% CO2 for 7 days. Evaluated the stability of non-replicating nanoparticles with the human lactoferrin gene insert contained in the composition by the change of their titres (evaluated in units of activity by the presence of clotting).

TABLE 7

| | Exposure time, min | | |
|---|---|---|---|
| Solvent or control substance | 0 | 30 | 60 |
| 0.9% sodium chloride | 1 × 10⁰ | 1 × 10⁰ | 1 × 10⁰ |
| Water for injections | 1 × 10⁰ | 1 × 10⁰ | 1 × 10⁰ |
| 5% glucose | 1 × 10⁶ | 1 × 10⁶ | 1 × 10⁶ |
| 10% glucose | 1 × 10⁶ | 7.3 × 10⁵ | 6.5 × 10⁵ |
| Cultural medium (control | 1 × 10⁶ | 1 × 10⁶ | 1 × 10⁶ |
| substance) | | | |

Evaluation of results shown in Table 7 showed preservation of stability of non-replicating nanoparticles without significant loss of titres in 5% and 10% glucose solutions, as well as in the control substance. But in 0.9% sodium chloride solution and in water for injections the titres of non-replicating nanoparticles reduced to nil, which signifies lack of stability of non-replicating nanoparticles in them.

Thus, the obtained results allow recommending 5% and 10% glucose solution as the acceptable solvents for dilution and intravenous injection of the pharmacological composition.

Confirmation of safety of administration of developed composition

The data characterizing effect of the pharmaceutical composition diluted in 5% and 10% glucose solutions for blood coagulability of rats ex vivo are provided in Table 8. For the experiment 3 ml of the composition with content 2.33×1011 v.p. per ml was dissolved in 200 ml of the solvent, the 80-fold dilution was obtained, which corresponded to 2.9×108 v.p. per ml. The non-stabilized rat blood was used in the study. The samples were incubated in the thermostat at a temperature 370C. Samples were visually evaluated at close observation of samples for 3 minutes.

TABLE 8

| | | Tested substance | | |
|---|---|---|---|---|
| | | | | Sodium |
| | | | | chloride |
| | | Pharmaceutical | Pharmaceutical | 0.9% |
| | Non-stabilized | composition + | composition + | (control |
| | venous blood | 5% glucose | 10% glucose | substance) |
| Time of blood | 368.00 ± 15.31 | 427.33 ± 11.56 | 392.67 ± 27.09 | 361.67 ± 15.31 |
| coagulation, sec | | | | |

Results represented in Table 8 show that the pharmaceutical composition dissolved by 5% or 10% glucose solution is well mixed with blood and doesn't lead to clotting. The time of blood coagulation of experimental samples as well as 0.9% sodium chloride solution (control substance) was comparable and reached 427.33±11.56; 392.67±27.09 and 361.67±9.28 seconds, respectively. The time of native blood coagulation made 368.00±15.31 seconds.

Thus, injection of the pharmaceutical composition in specified concentrations and the formulating buffer in the whole non-stabilized venous blood of rats didn't cause changes in samples that could signify incompatibility of this pharmaceutical composition and the buffer solution with blood.

### EXAMPLE 11

Determination of the way of administration of the pharmaceutical composition to a man

Previous experimental studies of the pharmaceutical composition were conducted on mice having no pre-existing immune response to the human adenovirus. But many people are known to have the pre-existing immune response to human adenoviruses as a result of natural contamination. A powerful immune response quickly neutralizes introduced constructions, which might considerably reduce the therapeutic medication effect. (Harvey B.-G., Hackett N. R., El-Sawy T. et al. Variability of human systemic humoral responses to adenovirus gene transfer vectors administrated to different organs. J. Virol, 1999, v. 73, pp. 6729-6742).

The effectiveness of the claimed scheme of the pharmaceutical composition way of administration with respect to removal of the pre-existing immune response was established in experiments on mice with artificially induced pre-existing immune response to human adenovirus 5 (Ad5).

The mice of line Balb/c with the weight 7-9 g were intramuscularly immunized by three injections of human adenovirus 5 without the lactoferrin gene insert at a dosage of 3×1011 v.p. per mice for receipt of the pre-existing immune response model. The level of virus-neutralizing antibodies to human adenovirus 5 was determined in three weeks after immunization. The antibody titre to the human adenovirus following intramuscular injection to mice reached 1:64 and was different from the control group (1:8—non specific titres).

The claimed scheme the pharmaceutical composition administration is based on peculiarities of the immune system allowing after the primary introduction of the specific antigen and its binding with pre-existing antibodies not to form new antibodies in response to repeated stimulation by the specific antigene. The effectiveness of the claimed scheme of the pharmaceutical composition administration was confirmed experimentally. The first group of mice with the induced pre-existing immune response to the human adenovirus 5 received a single intravenous injection recommended in previous studies on specific activity the full dosage of the pharmaceutical composition—4.3×1011 v.p. per sqm allowing achieving the maximum blood lactoferrin concentration (Cmax=3.6 μg per ml) on Day 6 after injection. The second group of mice with pre-existing immune response to human adenovirus 5 received partial dosage of the composition (4.3×1011 v.p. per sqm): on Day 1—one third (1.43×1011 v.p. per sqm), in a day—the full dosage of the pharmaceutical composition (4.3×1011 v.p. per sqm). Two more group of mice that didn't have the pre-existing immune response to human adenovirus 5 were formed for control, one group received the pharmaceutical composition containing the dosage 4.3×1011 v.p. per sqm, the other—an an equivalent volume of the NaCl physiological solution. All experimental and control groups included 5 animals.

Blood serums from all mice were collected on Day 6 and the human lactoferrin was determined in them in order to register the achieved effect on removal of pre-existing immune response (Table 9).

TABLE 9

| | | | | | Concentration |
|---|---|---|---|---|---|
| | | Experimental | Dosage, | Scheme | lactoferrin |
| Group | | or control | v.p. | of | a, |
| No. | Group name | substance | per sqm | administration | μg/kg |
| 1 | Pre-existing | Pharmaceutical | 4.3 × 10¹¹ | A single | 0.8 ± 0.12 |
| | response to Ad5 | composition | | injection | |
| 2 | Pre-existing | Pharmaceutical | 1.43 × 10¹¹ + | A double | 3.58 ± 0.51 |
| | response to Ad5 | composition | 4.3 × 10¹¹ | partial | |
| | | | | injection | |
| | | | | in a day | |
| 3 | No pre-existing | Pharmaceutical | 4.3 × 10¹¹ | A single | 3.6 ± 0.4 |
| | response to Ad5 | composition | | injection | |
| 4 | No pre-existing | 0.9% NaCl | 3 ml | A single | 0 |
| | response to Ad5 | solution | | injection | |
| | | (control | | | |
| | | substance) | | | |

Results of the experiment shown in Table 9 demonstrated that intravenous injection of one third from the full dosage of the pharmaceutical composition (1.43×1011 v.p. per sqm) effectively removes the pre-existing response to human adenovirus 5. This allowed achieving after the intravenous injection of the full dosage of the composition in a day constituting 4.3×1011 v.p. per sqm expression of the human lactoferrin in a therapeutic concentration (3.58±0.51μg/kg) on Day 6 after administration of the pharmaceutical composition, which is comparable to concentration of the recombinant lactoferrin obtained after a single administration in the full dosage to mice without the pre-existing immune response. In mice with pre-existing immune reaction a single injection of the composition in the full dosage didn't allow achieving the high concentration of the recombinant lactoferrin in the blood serum (0.8±0.12 μg/kg).

Thus, the scheme of administration of the pharmaceutical composition based on prior intravenous injection of one third of the major dosage (1.43×1011 v.p. per sqm) and subsequent intravenous injection of the full dosage of the composition in a day (4.3×1011 v.p. per sqm) allows achieving the therapeutic concentration of the recombinant lactoferrin in the body with pre-existing immune response to the human adenovirus 5.

Then in clinical examples dosages obtained during preclinical trials and measures per sqm of the body surface were recalculated per a man as they are equivalent (the average surface of the human body surface is equal to 1.62 sqm). (Khabriev R. U. Guidance on Experimental (Preclinical) Study of New Pharmacological Substances, 2000, p. 98) (Guidance for Industry. Estimating the Maximum Safe Starting Dose in Initial Clinical Trials for Therapeutics in Adult Healthy Volunteers. U.S. Department of Health and Human Services Food and Medication Administration Center for Medication Evaluation and Research (CDER), July 2005, Pharmacology and Toxicology—pp. 7, 19)

### EXAMPLE 12

Determination of Individual Therapy

Patient L. Thyroid carcinoma Stage IV, Clinical Group II (T4N2aM 1). The following chemical and therapeutic therapy was indicated: Cyclophosplmnum, Vincristine, Doxorubicin. At the beginning of the therapy grave blood disorders—erythropenia, lymphopenia, as well as general weakness, nausea, dyspepsia. After the intravenous drop infusion of the pharmaceutical composition in 67 ml of 10% glucose solution at a dosage of 2.33×1011 v.p. and subsequent intravenous drop infusion of the full dose of the pharmaceutical composition in 200 ml of 10% glucose solution in a day (7×1011 v.p.) the hematological values were improved, nausea and dyspepsia were eliminated and the general state of the patient improved. Then preventive administration of the pharmaceutical composition continued in accordance with the same scheme in 24 hours before the start of the following chemotherapy course.

### EXAMPLE 13

Determination of Individual Therapy

Patient V. Lymphatic Sarcoma, Stage IV (generalized form), state after the surgical therapy. After the subsequent chemotherapy (Cyclophosphane) a moderate toxic liver injury, pneumonia, erythro- and lymphopenia. In order to remove the above-stated toxic effects intravenous drop infusion of the pharmaceutical composition in 67 ml of 5% glucose solution at a dosage of 2.33×10¹¹ v.p. and subsequent intravenous drop infusion of the complete dosage of the pharmaceutical composition in 200 ml of 5% glucose solution in a day (7×10¹¹ v.p.). Application of the pharmaceutical composition in accordance with this scheme contributed to arresting of pneumonia, liver injury and blood hemodynamic values.

### EXAMPLE 14

Determination of Individual Therapy

Patient P. Metachromous carcinoma of the right mammary gland T3N0 M0. State after mastectomy. In the beginning of the course of the post-surgical radiation therapy (a single dosage 2 Gr each day on the post-surgical cicatrix) a local radiation reaction as inflammatory skin reaction in the site of radiation was observed, the patient started feeling general reactions—disturbance of the GIT (appetite reduction, nausea, vomiting, diarrhea). Intravenous drop infusion of the pharmaceutical composition in 67 ml of 10% glucose solution at a dosage of 2.33×1011 v.p. and subsequent intravenous drop infusion of the full dosage of the pharmaceutical composition in 200 ml of 10% glucose solution in a day (7×1011 v.p.) were applied for removal of the above-stated toxic effects. Application of the pharmaceutical composition in accordance with this scheme allowed improving the general state of the patient and to remove inflammatory reactions in the site of radiation as well. AST, ALT values, creatinine and urea are in norm.

As a result, the claimed pharmaceutical composition contains human adenovirus 5 genome based non-replicating nanoparticles with the human lactoferrin gene insert allowing after injection at dosages from 7×1011 v.p. to 7×1013 v.p. per person achieving prolonged (from 12 hours to 29 days) human lactoferrin expression. The human lactoferrin expressed by the pharmaceutical composition corresponds to the native lactoferrin from the donor human milk by physicochemical properties, antioxidant activity and antimicrobial properties. The detoxifying action of the pharmaceutical composition is achieved after injection at dosages from 7×1011pp to 7×1012 v.p. per person. A pharmaceutical composition with two dosage forms—1 ml and 3 ml with content of non-replicating nanoparticles at a dosage of no less than 2.33×1011 v.p. per ml is claimed for implementation of the claimed way of therapy. In accordance with the claimed way of therapy the first dosage of the pharmaceutical composition is administered to a patient through intravenous drop infusion at a dosage of 2.33×1011 v.p. contained in 1 ml of the composition preliminary dissolved by 67 ml of 5% or 10% glucose solution. The second administration of the composition is done in a day—the full dosage 7×1011 v.p. contained in 3 ml of the composition is preliminarily dissolved by 200 ml of 5% or 10% glucose solution and is administered through intravenous drop infusions. The claimed method of therapy allows treating various etiology toxicoses safely and effectively in patients with pre-existing immune response to human adenovirus 5, which demonstrates achievement of tasks set in this invention.

Thus, as compared to the prototype the advantages of the claimed pharmaceutical composition [symbol] non-replicating nanoparticles producing the antioxidant human protein—lactoferrin are:

a single administration for obtainment of the therapeutic concentration in the human body;

prolonged effect (up to 3 weeks);

reduction of medication and medical instruments consumption, working time of medical personnel;

removal of the necessity to use human milk for production of lactoferrin;

reduction of production costs;

a possibility to receive a relatively greater lactoferrin amounts.

Thus, the set technical task targeted at development of the pharmaceutical composition on the basis of non-replicating nanoparticles, producing immediately in the body the antioxidant human protein—lactoferrin with antioxidant, antimicrobal and antitoxic protein, suitable for therapy of toxic states of various etiology and providing achievement of the stable therapeutic effect of this composition was fulfilled. Application of the claimed pharmaceutical composition provides reduction of medication and medical instruments consumption as well as of the time of medical personnel in order to achieve the required therapeutic result.

Use in the pharmaceutical and clinical practice of the claimed pharmaceutical composition and methods of its application allows achieving several technical, therapeutic and economic results:

the claimed pharmaceutical composition is biocompatible with the human body and is highly effective from the therapeutical point of view;

non-replicating nanoparticles producing antioxidant human proteins are compatible to various carriers—solvents for intravenous use;

a pharmaceutical composition is suitable for therapy of various localization diseases;

the pharmaceutical composition is convenient for use as it's administered as a single injection and then for a long time (3 weeks) it produces in the human body a biologically active antioxidant human protein creating in the blood concentration that by ten-fold exceeds the normal value and required for the stable therapeutic effect;

application of the pharmaceutical composition is economically justified as a single injection of the medication ensures a long-term therapeutic effect.

What is claimed is:

1. A pharmaceutical composition producing an antioxidant, antimicrobal, antitoxic Protein—human lactoferrin, the composition comprising:
non-replicating nanoparticles based on a genome of human adenovirus 5 genome with an insert of a human lactoferrin gene, the nanoparticles expressing the human lactoferrin in a therapeutically effective amount in a human body; and
a formulating buffer;
wherein the therapeutic effect of the pharmaceutical composition is achieved as a result of an action of the antioxidant, antimicrobal, antitoxic protein - human lactoferrin on the human body.

2. The pharmaceutical composition of claim 1, wherein a concentration of the non-replicating nanoparticles is no less than 2.33×10″ virus particle (v.p.) per ml of the formulating buffer.

3. A method of producing a pharmaceutical composition comprising:
providing non-replicating nanoparticles based on a genome of human adenovirus 5 with an insert of an expressing cassette including a promoter, a human lactoferrin gene and a polyadenylation signal;
achieving a target activity by seeding a permissive cell culture 293 with non-replicating nanoparticles carrying the human lactoferrin gene;
growing the non-replicating nanoparticles in a cell until they achieve a desired concentration;
multi-step purifying of a solid part obtained at the growing step comprising:
centrifuging, four-fold freezing-thawing it in a buffer solution;
treating by a nuclease;
isolating of the non-replicating nanoparticles carrying the human lactoferin gene from damaged cells by centrifuging and subsequent separation of an obtained supernatant;
subsequently purifying the obtained supernatant by ultrafiltration comprising dissolving the obtained supernatant by the buffer and stirring followed by filtration under pressure;
purifying by anion-exchange chromatography followed by size exclusion chromatography; and
adding ethanol and ethylenediamine-tetraacetic acid to an obtained eluate followed by regular filtration; and
obtaining the pharmaceutical composition by dissolving a product obtained at the previous step in a formulating buffer until a predefined amount of the non-replicating nanoparticles is produced.

4. The method of claim 3, wherein further producing the non-replicating nanoparticles based on a genome of human adenovirus 5 genome with an insert of a human lactoferrin gene by a homologous recombination method in the cell culture.

5. The pharmaceutical composition of claim 1, wherein a therapeutically effective dosage of the non-replicating nanoparticles is taken per 3 ml of a final volume of the composition.

6. The pharmaceutical composition of claim 5, wherein a dosage form of a medication comprising the composition is 1 ml.

7. The pharmaceutical composition of claim 5, wherein a dosage form of a medication comprising the composition is 2 ml.

8. The pharmaceutical composition of claim 5, wherein a dosage form of a medication comprising the composition is 3 ml.

9. A therapeutical method comprising administering a therapeutically effective amount of a pharmaceutical composition producing a antioxidant, antimicrobal, antitoxic protein—human lactoferrin to a patient.

10. The method of claim 9, wherein administering the pharmaceutical composition comprises injecting the composition intravenously.

11. The method of claim 9, wherein administering the therapeutically effective amount of the pharmaceutical composition comprises intravenous drop infusion injection.

12. The method claim 9, wherein the pharmaceutical composition is administered to treat toxic states caused by pyoinflammatory diseases induced by microorganisms, physical exposures and chemical agents, drug therapy and radiation therapy.

13. The method of claim 9, wherein a therapeutically effective dosage of non-replicating nanoparticles based on a genome of human adenovirus 5 genome with an insert of a human lactoferrin gene ranges from 7×10″ virus particle (v.p.) to 7×10¹³ virus particle (v.p.) per patient.

14. The method claim 11, wherein the therapeutically effective amount of the pharmaceutical composition is injected sequentially as two stages.

15. The method claim 14, wherein injecting the therapeutically effective amount of the pharmaceutical composition at a second stage is administered a day after a first administration of the composition.

16. The method of claim 14, wherein administering the therapeutically effective amount of the pharmaceutical composition at a first stage comprises injecting ⅓ of a volume of a therapeutical dosage, and injecting ⅔ of the volume of the full therapeutical dosage at a second stage.

17. The method of claim 16, wherein the ⅓ of the volume of the full therapeutical dosage of the pharmaceutical composition is dissolved in 66 ml of a physiologically acceptable solvent, and wherein ⅔ of the volume of the full therapeutical dosage of the pharmaceutical composition are dissolved in 134 ml of the physiologically acceptable solvent.

18. The method of claim 17, wherein the physiologically acceptable solsolvent is a glucose solution.

19. The method of claim 18, wherein the physiologically acceptable solvent is a 5% glucose solution.

20. The method of claim 18, wherein the physiologically acceptable solvent is a 10% glucose solution.
